(19) United States
(12) Patent Application Publication
(10) Pub. No.: US 2003/0031703 A1
(43) Pub. Date: Feb. 13, 2003

(54) Textured article

(76) Inventors: Linda J. McMeekin, Bound Brook, NJ (US); Joseph Luizzi, Newtown, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/923,552

(22) Filed: Aug. 7, 2001

(51) Int. Cl. A61K 9/70; B32B 27/42

(52) U.S. Cl. 424/443; 442/161

(57) ABSTRACT

An article containing: a substrate having at least two surfaces; and raised elements on at least one surface of the substrate, wherein the article is useful for providing skin care benefits is disclosed. The raised elements may be made from hot melt coatings, natural rubber, synthetic rubber, polyolefins, ethylene vinyl acetate, or thermoplastic elastomers.

### BACKGROUND OF THE INVENTION

1. Field of the Invention

The present invention relates to a textured article. The article contains raised elements on at least one surface. The raised elements provide gentle massaging, cleansing, scrubbing and/or exfoliating action for skin, hair or surface treatment. The article may contain an active that is intended to be applied to, or interact with, the surface of the skin or hair. The substrate for the article may be a woven or knit fabric, a nonwoven, a laminate containing a fabric and a polymeric film, a flocked fabric, and combinations thereof.

2. Description of the Prior Art

In recent years, many articles have been developed to aid in wiping various substrates. One example is the development of baby wipes, which are used to wipe the baby clean during diaper changes. Baby wipes typically are soft and are loaded with a cleanser and moisturizer. Baby wipes are not known for exfoliating properties. As used herein, exfoliating means removing dead skin from the surface of the skin.

Another type of wipe that has been developed is the hand wipe. Hand wipes are used to clean the hands when the use of a sink is inconvenient. These wipes typically are not as soft as baby wipes. Hand wipes frequently contain both cleansers and antibacterial agents. Hand wipes are not known to have exfoliating properties.

Wipes have also been developed for cleaning the face. These wipes are typically very soft and contain cleansers, moisturizers, and anti-acne agents. Most face wipes are not known for exfoliating properties. The BUFF-PUFF® pad is sold commercially for exfoliating and cleansing the face. The pad is made from a spun-bond polymer and has a very rough texture. Although the pad is effective at cleansing and exfoliating the skin, some consumers find the pad to be too rough. Therefore, there is a need for a wipe that cleans and exfoliates the skin without being too rough on the skin.

It is known to selectively place rubbery materials on a cloth surface to improve friction and reduce slip. For example, TOTES® slipper socks are made of a fabric that would be slippery on floor surfaces. To overcome this problem, the fabric is treated to place raised rubbery elements in the form of dots on the surface of the fabric that contacts the floor.

Another example of selectively placing materials on a cloth substrate is disclosed in U.S. Pat. No. 5,538,732. The patent teaches selectively placing dots of water soluble active ingredients on wipes. In use, the wipes are wetted, then applied to the skin to deliver the active ingredients. The dots are not raised and do not exfoliate or massage the skin.

Despite the disclosure of the prior art, there remains a need for a wipe that cleans and exfoliates the skin without being too rough on the skin.

### SUMMARY OF THE INVENTION

The present invention provides an article including a substrate having at least two surfaces; and raised elements on at least one surface of the substrate, wherein the article is useful for providing skin or hair care benefits.

### DETAILED DESCRIPTION OF THE PREFERRED EMBODIMENT

The article of the invention includes a substrate. Suitable substrates are known in the art of wipes and include, but are not limited to, a woven fabric, a knit fabric, a nonwoven fabric, a laminate of a fabric and a polymeric film, such as a polyolefin film, a flocked fabric, and combinations thereof. Methods of making woven and knit cloths are not a part of this invention and, being well known in the art, are not described in detail herein. One type of nonwoven cloth substrate utilized in the present invention is made by air- or water-laying processes in which the fibers or filaments are first cut to desired lengths from long strands, passed into a water or air stream, and then deposited onto a screen through which the fiber-laden air or water is passed. The deposited fibers or filaments are then adhesively bonded together, and otherwise treated as desired to form the woven, nonwoven, or cellulose cloth.

In another embodiment, the substrate utilized in the present invention may be a thermal bonded nonwoven cloth (whether or not resin-containing) which can be made of polyesters, polyamides, polyolefins, or other thermoplastic fibers which can be spun bonded, i.e., the fibers are spun out onto a flat surface and bonded (melted) together by heat or chemical reactions.

When nonwoven substrates are utilized,-the nonwoven cloth substrates are generally adhesively bonded fibers or filamentous products having a web or carded fiber structure (when the fiber strength is suitable to allow carding) or comprising fibrous mats in which the fibers or filaments are distributed haphazardly or in random array (i.e., an array of fibers in a carded web where partial orientation of the fibers is frequently present, as well as a completely haphazard distributional orientation), or substantially aligned. The fibers or filaments can be natural (e.g., wool, silk, jute, hemp, cotton, linen, sisal, or ramie) or synthetic (e.g., rayon, cellulose ester, polyvinyl derivatives, polyolefins, such as polyethylene and polypropylene, polyamides, such as nylon 6, nylon 6,6, or polyesters, such as polyethylene terephthalate and polybutylene terephthalate), or combinations thereof. These nonwoven materials are generally described in the INDA “NONWOVEN FABRICS HANDBOOK”, (1999), hereby incorporated by reference for nonwoven substrates and their methods of manufacture. The basis weight of the substrate may vary, but generally ranges from about 20 grams per square meter to about 500 grams per square meter, for example from about 50 grams per square meter to about 150 grams per square meter.

The substrate has at least two surfaces, generally a top surface and a bottom surface. The article of the invention is useful for exfoliating skin, therefore the article contains raised elements on at least one surface of the substrate. The raised elements may be discrete.

The raised elements are made of any suitable material for providing abrasive or massaging properties. Suitable materials include, but are not limited to, hot melt coatings, natural rubber, synthetic rubber, polyolefins, such as polyethylene and polypropylene, ethylene vinyl acetate, and thermoplastic elastomers. Colorants or pigments may be combined with the coating materials.

Suitable hot melt coatings for generating raised elements include HL-7471 W from H. B. Fuller Co., St. Paul, Minn., and REXTAC amorphous polyolefins, available through Huntsman Chemical. For example, hot melt coatings containing from about 15% to about 100% olefin polymer or a block copolymer, from about 0% to about 60% tackifying resin, and from about 0% to about 50% wax may be useful. Suitable olefin polymers include polymers:

a) wherein the olefin polymer is a homopolymer of ethylene, propylene, n-butene, butylene or isobutylene, with a melt flow index from 0.5 to 2500, such as Ateva™ polymers from AT plastics; Escorene® and Vistanex® polymers from Exxon Chemical, Duraflex® polymers from Shell Chemical, Epolene® polymers from Eastman Chemical, and Vestoplast® polymers from Creanova;

b) wherein the olefin polymer is a copolymer of ethylene and a co-monomer, such as vinyl acetate, acrylic acid, methacrylic acid, ethyl acrylate, methyl acrylate, n-butyl acrylate vinyl silane or maleic anhydride, such as Ateva™ polymers from AT plastics, Elvax® polymers from DuPont, Escorene® and Optema® polymers from Exxon Chemical, and Primacor® polymers from Dow Chemical; and

c) wherein the olefin polymer is a terpolymer of ethylene and co-monomers, such as vinyl acetate, acrylic acid, methacrylic acid, ethyl acrylate, methyl acrylate, n-butyl acrylate vinyl silane or maleic anhydride, such as Ateva™ polymers from AT plastics, Nucrel® polymers from DuPont, and Escor® polymers from Exxon Chemical.

Suitable block copolymers include block copolymers having a linear or a radial structure such that the structure (A—B)ₓ where A is consists essentially of a polyvinylarene block, and B consists essentially of poly(monoalkenyl) block, and x denotes the number of polymeric arms, where x is greater than or equal to one are also useful. Block B may be selected from conjugated diene elastomers such as polybutadiene or polyisoprene and hydrogenated elastomers such as ethylene-butylene or ethylene-propylene. Suitable examples of these types of polymers include Kraton® elastomers from Shell Chemical Company, Vector® elastomers from Dexco, Solprene® elastomers from Enichem Elastomers and Stereon® from elastomers Firestone Tire & Rubber Co. When the hot melt coatings contain block copolymers, it is preferable for the coating to contain from about 15% to about 50% block copolymer.

Suitable tackifying resins include any compatible resin or mixture thereof selected from the group consisting of a) natural and modified rosins; b) glycerol and pentaerythritol esters of natural and modified rosins; c) polyterpene resins; d) copolymers and terpolymers of natural terpenes; e) phenolic modified terpene resins and the hydrogenated derivatives thereof, f) aliphatic petroleum resins and the hydrogenated derivatives thereof; g) aromatic petroleum resin and the hydrogenated derivatives thereof; and h) aliphatic/aromatic petroleum resins and the hydrogenated derivatives thereof, such as Foral® resin, Staybelite® resin, Poly-pale® resin, Permalyn® resin, Pentalyn® resin, Adtac® resin, Piccopale® resin, Piccotac® resin, Hercotac® resin, Regalrez® resin, and Piccolyte® resin from Hercules, Escorez® resin from Exxon Chemical, Wingtack® resin from Goodyear Tire & Rubber Co., Arkon® resin from Arakawa Chemicals, Zonatac® resin, Zonarez® resin and Zonester® resin from Arizona Chemical and Nevtac® resin from Neville Chemical Company.

Suitable waxes include, but are not limited to, paraffins, Fischer-tropsh, and microcrystalline waxes, and combinations thereof. Suitable microcrystalline waxes include, but are not limited to, BE SQUARE 175 microwax, available from Bareco Division, Petrolite Corporation, and M-5165 from Moore & Munger, Shelton, Conn.

Suitable polyethylene waxes include, but are not limited to, H-101 from Exxon Chemical, Houston, Tex. Suitable Fischer-Tropsch waxes include, but are not limited to, Paraflint Wax from Schumann Sasol, Hamburg, Germany.

The raised elements may be applied onto the substrate by any means known in the art, such as control coating, control fiberization, pattern coating, gravure coating, rotary screen printing, and spray coating. Equipment for coating the substrates is commercially available. One example is the DYNAFIBER, available through Nordson Company. Another example is the ITW, available through Omega Company. When applying raised elements through a melt process, the time it takes to cool the applied coating affects the height of the raised elements. If the coating is not cooled quickly enough, the coating may penetrate the substrate to the extent that no raised element is formed. To overcome this problem, an air knife that utilizes air, which may be chilled, may be utilized to quickly cool the applied coating and prevent tailing. The angle of contact between the air and the applied coating may also affect the height of the raised elements. The air typically contacts the coating at an angle of from about 10 to about 80°.

The raised elements may be of any shape including, but not limited to, lines, waves, interconnected patterns, circular dots, hexagons, hearts, diamonds, rectangles, stars, triangles and the like. The density, height, and diameter of the raised elements may vary depending on the massaging or exfoliating properties desired. Generally, the raised elements may occupy anywhere from about 1 percent to about 99 percent, for example from about 10 percent to about 75 percent, or from about 20 percent to about 50 percent of the surface of at least one side of the substrate. The height of the raised elements, as measured from the surface of the substrate, should be sufficient to provide exfoliating or massaging properties. Generally, the height is at least 0.01 mm above the surface of the substrate.

When the raised elements are discrete, the elements have a diameter sufficient to provide exfoliating or massaging properties. Generally, the diameter of the discrete raised elements may range from about 0.5 mm to about 3 mm, for example about 1 mm to about 2 mm.

The articles of the present invention provide a skin care benefit or a hair care benefit. As used herein, skin care benefit and hair care benefit means massaging, cleansing, scrubbing and/or exfoliating the skin.

The articles of the invention are preferably combined, e.g., coated or impregnated, with cleansers, skin and/or hair care actives, moisturizers, and the like. The articles may be loaded with appropriate skin and/or hair care compositions and packaged as wet wipes. The skin care or hair care compositions may be loaded onto the wipes by dipping the wipes in the skin care composition, spraying the skin care composition onto the wipes, and other means known in the art.

Alternatively, the wet wipe may be dried through the use of heating equipment or vacuum driers to provide dry wipes. Alternatively, a cleansing or skin care formulation may be applied in the form of a concentrate to the substrate to provide dry textured articles. The dry textured articles or wipes are sold dry, then the consumer wets the wipe with water when ready for use.

Several examples are set forth below to further illustrate the nature of the invention and the manner of carrying it out. However, the invention should not be considered as being limited to the details thereof.

### EXAMPLE 1

A textured article was prepared on a Kramer Coater utilizing 80 gsm Jacob Holm (100% polyethylene terephthalate spunlace nonwoven) as the substrate and Fuller HL-7471W (with titanium dioxide) as the discreet raised elements. The HL-7471W was melted at 350° F. and pattern applied to the substrate using a random dot screen pattern number 50129 (Rothtec Engraving Corporation) at a coat weight of 34.4 g/m². The dot screen pattern had 146,500 dots per square meter. The circular dots had an average diameter of 1.28 mm, a height of 0.08 mm, and were spaced apart approximately 3.57 mm.

### EXAMPLE 2

A second textured article was prepared on a May Coater utilizing Orlandi 55 gsm (70% polyester/30% Rayon spunlace nonwoven) as the substrate and Fuller HL-7471W (with titanium dioxide) as the discreet raised elements. The HL-7471W was melted at 325° F. and pattern applied to the substrate at a coat weight of 38.06 g/yd² using a Rothtec random dot screen. The dot screen pattern had 146,500 dots per square meter. The circular dots had an average diameter of 1.36 mm, a height of 0.04 mm, and were spaced apart approximately 3.29 mm.

We claim:

1. A textured article comprising:
a substrate having at least two surfaces; and
raised elements on at least one surface of the substrate, wherein the article is useful for providing skin or hair care benefits.

2. The article according to claim 1 wherein the substrate is selected from the group consisting of a woven fabric, a knit fabric, a nonwoven fabric, a laminate of a fabric and a polymeric film, a flocked fabric, and combinations thereof.

3. The article according to claim 1 wherein the raised elements are formed from hot melt coatings comprising a polymer selected from the group consisting of olefin polymers, block copolymers, and combinations thereof.

4. The article according to claim 3 wherein the hot melt coating comprises from about 15% to about 100% olefin polymer or a block copolymer, from about 0% to about 60% tackifying resin, and from about 0% to about 50% wax.

5. The article according to claim 1 wherein the raised elements are discrete and are in a shape selected from circular dots, hexagons, hearts, diamonds, rectangles, stars, and triangles.

6. The article according to claim 2 wherein the substrate has a basis weight ranging from about 20 grams per meter to about 500 grams per square meter.

7. The article according to claim 5 wherein the discrete raised elements cover from about 10 percent to about 90 percent of the surface of at least one side of the substrate.

8. The article according to claim 6 wherein the discrete raised elements have a diameter ranging from about 0.5 mm to about 3 mm.

9. The article according to claim 6 wherein the discrete raised elements have a height greater than about 0.01 mm as measured from the surface of the substrate.

10. The article according to claim 1 further comprising a skin or hair care composition.

11. A method of providing an exfoliating wipe comprising:
providing a substrate selected from the group consisting of a woven fabric, a knit fabric, a nonwoven fabric, a laminate of a fabric and a polymeric film, a flocked fabric, and combinations thereof; and pattern coating raised elements onto the substrate utilizing a hot melt coating selected from the group consisting of olefin polymers, block copolymers, and combinations thereof.

12. The method according to claim 11 wherein the pattern coating is applied by a method selected from hot melt rotary screen coating and hot melt gravure coating.

13. A method for cleansing the skin or hair comprising topically applying the article of claim 1 to said skin or hair.

14. The article of claim 1 wherein the article is in the form of a dry or wet wipe.

15. A method for cleansing exfoliating or massaging the skin comprising topically applying the article of claim 1 to said skin.
